(19) United States
(12) Patent Application Publication
(10) Pub. No.: US 2020/0046737 A1
(43) Pub. Date: Feb. 13, 2020

(54) METHODS FOR TREATING CANCER, AND COMPOSITIONS THEREFOR

(71) Applicant: Notable Labs, Inc., Foster City, CA (US)

(72) Inventors: Kamran Ali, Foster City, CA (US); Nina Myers, San Mateo, CA (US); Christos Gekas, Foster City, CA (US); Diane Heiser, San Mateo, CA (US); Stephen Western, Vancouver, CA (US); Marianne Santaguida, Belmont, CA (US); Matt De Silva, Burlingame, CA (US)

(21) Appl. No.: 16/536,199

(22) Filed: Aug. 8, 2019

Related U.S. Application Data
(60) Provisional application No. 62/716,802, filed on Aug. 9, 2018.

(51) Int. Cl. A61K 31/593; A61K 31/573; A61K 31/202; A61K 31/4045; G01N 33/50

(52) U.S. Cl. CPC A61K 31/593; A61K 31/573; G01N 33/5011; A61K 31/4045; G01N 33/5023; A61K 31/202

(57) ABSTRACT

Provided herein are methods for treating proliferative diseases, such as cancer, via novel priming-eligible targets; and compositions therefor.

### CROSS-REFERENCE TO RELATED APPLICATIONS

Under 35 U.S.C. § 119(e), this application claims priority to the filing date of U.S. Provisional Patent Application No. 62/716,802, filed on Aug. 9, 2018, the disclosure of which application is herein incorporated by reference in its entirety.

### FIELD OF THE INVENTION

The field of the invention is related to novel methods of treating proliferative diseases.

### BACKGROUND OF THE INVENTION

Antibody based therapeutics provide highly targeted approaches for treating hematologic malignancies by specifically targeting therapeutic agents to cancerous cells that preferentially express a target surface marker.

Cancer target identification remains a major challenge in the development of antibody-based therapeutics as target surface markers require a wide “therapeutic window” in which the diseased cells express significantly higher amounts of the target than normal cells express. The therapeutic window is a range of doses that produces therapeutic response without causing any significant adverse effect in patients. Generally therapeutic window is a ratio between minimum effective concentrations (MEC) to the minimum toxic concentration (MTC). If this criterion is not met, these agents can cause significant toxicity in healthy tissue. Few surface markers satisfy these criteria since most surface markers provide some type of normal function to healthy tissues and are generally expressed normally at some level. Current approaches for identifying adequate candidate surface markers have had limited success translating to the clinic: of the hundreds of clinical trials for antibody drug conjugates (ADCs), only 4 have been approved with many trials failing due to toxicity issues.

Accordingly, new cancer targets or surface markers and methods for identifying and therapeutically targeting them are needed.

### SUMMARY OF THE INVENTION

Provided herein are novel targets (“primed targets”) for treating proliferative disease, such as cancer, and the like. Also provided herein are methods for identifying novel primed targets for therapeutic intervention and treating proliferative diseases.

In a particular embodiment, provided herein is an ex vivo flow cytometry platform for high throughput testing of therapeutic agents on patient malignancies that provides a fundamentally different approach for identifying targets for therapeutics, such as immunotherapy or antibody-based therapeutics, and the like.

The invention methods for target generation for therapeutics, e.g., antibody-based therapeutics, and the like, circumvents the standard target identification methodology by utilizing “priming agents” that cause an increase in a specified target (e.g., a surface or intracellular marker) expressed on or within cancerous cells but not healthy cells. This increased expression of the specified primed target advantageously increases the therapeutic window by increasing the volume of primed targets, for example, a cell surface marker, such as CD126, CD44, CD24, CD15, and the like, available to any therapeutic agent that binds to said target.

Using the invention methods provided herein, “primed surface markers” or primed targets that previously did not have an adequate therapeutic window are newly provided herein as druggable immunotherapy targets (e.g., via antibody therapeutics, and the like) for treating cancer. In accordance with the present invention, priming agents are used to proactively cause an increase in the expression of these markers or primed targets (cell-surface or intracellular markers) in cancerous cells compared to healthy cells. These primed targets or primed surface markers are then used, in accordance with the present invention in therapeutic methods, as targets for a variety of therapeutic agents or modalities, including, for example, antibody based therapeutics such as a chimeric antigen receptor T-cell therapy (CAR-T), monoclonal antibody (mAb) therapy, antibody-dependent cell-mediated cytotoxicity (ADCC) mediated antibodies, bispecific antibodies, antibody-drug conjugates (ADCs), and the like.

Accordingly, provided herein are methods for treating a proliferative disease, such as cancer and the like, in a subject in need thereof, comprising: priming the subject with a priming agent to increase the level of a specified target; and administering to the subject an effective amount of a therapeutic agent that binds to the specified target.

In some embodiments of the claimed methods, the proliferative disease is cancer. In certain embodiments, the cancer is hematopoietic cancer. In some embodiments, the hematopoietic cancer is leukemia or lymphoma. In certain embodiments, the leukemia is acute lymphocytic leukemia (ALL), acute myelocytic leukemia (AML), chronic myelocytic leukemia (CML), or chronic lymphocytic leukemia (CLL). In some embodiments, the lymphoma is Hodgkin lymphoma (HL), non-Hodgkin lymphoma (NHL), follicular lymphoma, chronic lymphocytic leukemia/small lymphocytic lymphoma (CLL/SLL), mantle cell lymphoma (MCL), marginal zone B-cell lymphoma, primary mediastinal B-cell lymphoma, Burkitt lymphoma, lymphoplasmacytic lymphoma, hairy cell leukemia (HCL), immunoblastic large cell lymphoma, precursor B-lymphoblastic lymphoma, or primary central nervous system (CNS) lymphoma.

In certain embodiments of the claimed methods, the priming agent is selected from any combination of one or more of Dexamethasone; Tretinoin; Calcitriol; and Panobinostat. In certain other embodiments, the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat.

In certain embodiments of the claimed methods, the target is selected from the group consisting of: CD126, CD44, CD24, and CD15.

In some embodiments, the target is CD126, and the priming agent is selected from any combination of one or more of Dexamethasone; Tretinoin; Calcitriol; Panobinostat. In certain of those embodiments, the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat. In certain other embodiments, the priming agent is a combination of Dexamethasone and Tretinoin.

In other embodiments, the target is CD44, and the priming agent is selected from any combination of one or more of the following agents, selected from Dexamethasone; Tretinoin; Calcitriol; and Panobinostat. In certain of those embodiments, the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat.

In other embodiments, the target is CD24, and the priming agent is selected from any combination of one or more of Dexamethasone; Tretinoin; Calcitriol; and Panobinostat. In certain of those embodiments, the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat. In other embodiments, the priming agent is a combination of Dexamethasone and Tretinoin.

In some embodiments, the target is CD15, and the priming agent is selected from any combination of one or more of Dexamethasone; Tretinoin; Calcitriol; and Panobinostat. In certain of these embodiments, the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat. In other embodiments, the priming agent is a combination of Dexamethasone and Panobinostat; or a combination of Tretinoin and Panobinostat.

In certain embodiments of the claimed methods, the priming agent is a priming-tuning agent, which additionally functions to suppress expression of at least one undesired target surface protein, such as a target surface protein antagonizing T-cells, e.g. PD-L1.

In some embodiments, the methods of the invention further comprise administering to the subject a priming-tuning agent which additionally functions to suppress expression of at least one undesired target surface protein, such as a surface marker antagonizing T-cells, e.g. PD-L1. In some embodiments, the priming-tuning agent is Panobinostat. In certain embodiments, Panobinostat is administered in combination with Dexamethasone or Tretinoin.

Other aspects of the invention provide methods for identifying druggable therapeutic targets, comprising a) contacting a test-cancerous-cell, and a non-cancerous-control-cell, with one or more priming agents; b) assaying marker expression on both the test-cancerous-cell and the non-cancerous-control-cell in response to the priming agent, and further assaying surface marker expression on a control-cancerous-cell that was not contacted with priming agents; and c) identifying as a candidate therapeutic target any markers that have increased expression in the test-cancer-cell compared to both the control-cancerous-cell and the non-cancerous-control-cell. In some embodiments, the marker expression is selected from extracellular or intracellular expression.

Other aspects of the invention provide methods for identifying druggable targets, comprising a) contacting a test-cancerous-cell, and a non-cancerous-control-cell, with one or more priming agents; and b) assaying surface marker expression on both the test-cancerous-cell and the non-cancerous-control-cell in response to the priming agent, and further assaying surface marker expression on a control-cancerous-cell that was not contacted with priming agents; wherein increased surface marker expression of a particular target on the test-cancerous-cell compared to both the control-cancerous-cell and the non-cancerous-control-cell, identifies a druggable target.

Other aspects of the invention provide methods for identifying priming agents comprising a) contacting a test-cancerous cell and a non-cancerous cell with a plurality of priming agent candidates; b) assaying surface marker expression on both the test-cancerous cell and the non-cancerous cell in response to the priming agent candidates, and further assaying surface marker expression on a control-cancerous cell that was not contacted with priming agent candidates; and c) identifying priming agent candidates that increase surface marker expression on the test-cancerous cell compared to both the control-cancerous cell and the non-cancerous-control cell as priming agents. In certain embodiments, the claimed methods further comprise administering one or more priming agents identified to a subject in need. In some embodiments, the subject in need has a proliferative disease. In certain embodiments, the proliferative disease is cancer.

In other aspects, the invention provides a priming agent selected from any combination of one or more of Dexamethasone, Tretinoin, Calcitriol, and Panobinostat for use in the treatment of a proliferative disease and kits comprising one or more claimed priming agents. In some embodiments, the proliferative disease is cancer. In certain embodiments, the cancer is a hematological cancer. In some embodiments, the priming agent is Dexamethasone and Tretinoin; Dexamethasone and Panobinostat; Tretinoin and Panobinostat; Dexamethasone and Tretinoin and Calcitriol; or Dexamethasone and Tretinoin and Panobinostat.

In certain embodiments, the kits of the claimed invention further comprise a therapeutically active pharmaceutical composition. In one embodiment, the kit of the invention comprises the priming agent(s) in a first container and the therapeutically active pharmaceutical composition in a second container. In some embodiments, the therapeutically active pharmaceutical composition is to treat a proliferative disease. In one embodiment, the proliferative disease is cancer. In certain embodiments, the cancer is a hematological cancer.

Also provided herein, are therapeutic compositions comprising an ADC, CAR-T, mAb, or a Bispecific T-cell engaging (BiTE) antibody, that specifically bind to a target selected from CD126, CD44, CD24, CD15, and the like, as described herein, and optionally a pharmaceutically acceptable excipient. In certain embodiments, as described herein, the pharmaceutical composition of the invention comprises an ADC comprising an antibody selected from the group consisting of an: anti-CD126, anti-CD44, anti-CD24, and an anti-CD15 antibody; a linker, such as 4-thiopentanoate or the like; and a suitable toxin, such as maytansinoid, or the like; and optionally a pharmaceutically acceptable excipient, and their use in treating a proliferative disease, such as cancer.

### BRIEF DESCRIPTION OF THE DRAWINGS

FIGS. 1A and 1B shows a comparison of a priming agent and vehicle control Dimethyl Sulfoxide (DMSO) on healthy patient white blood cells and acute myeloid leukemia (AML) patient blast cells.

A) Healthy white blood cells from 3 donors show little increase in target surface marker expression in response to priming agents. (B) Percentage of blasts positive for the target surface markers increases in response to priming agents for three surface markers. [Blue: DMSO Treatment, Orange: Priming Compound Treatment]

FIG. 2 shows the results of Priming Tuning of CD15 in AML.

FIG. 3 shows the results of Priming of CD15 in multiple AML patients.

FIG. 4 shows the results of Priming CD126 in Patient 1 with various agents.

FIG. 5 shows the results of Priming CD24 in Patient 1 with various agents.

FIG. 6 shows the results of Priming CD44 in Patient 1 with various agents.

FIG. 7 shows the combined results of Priming CD126, CD24 and CD44 in Patient 1 with Dexamethasone and Tretinoin.

### DETAILED DESCRIPTION OF THE INVENTION

Provided herein are novel targets (e.g., primed targets) for treating proliferative disease, such as cancer, and the like. Also provided herein are methods for identifying novel primed targets for therapeutic intervention and treating proliferative diseases. Also, provided herein is a method for treating a proliferative disease, such as cancer and the like, in a subject in need thereof, comprising: priming or contacting the subject with a priming agent to increase the level of a specified target (thereby generating a “primed target”); and administering an effective amount of a therapeutic agent that binds to the specified primed target.

As used herein, the phrase “primed target,” “priming targets,” “priming-eligible target” or grammatical variations thereof, refers to an intracellular or extracellular molecule (e.g. a protein) whose expression or presence can be, or has been, upregulated or increased to become druggable by virtue of the cell that expresses the particular target(s) coming in contact with one or more priming agent(s), in accordance with the present invention. In particular embodiments, the priming agent or priming agents can increase or upregulate more than one priming-eligible target, such that a single priming treatment regimen can give rise to multiple primed targets, whose increased presence or expression are collectively susceptible to therapeutic intervention by one or more therapeutic agents that bind the respective primed target(s).

As used herein, the term “priming” generally refers to contacting the cells with one or more agents to sensitize the cells (e.g., cancer cells), by proactively upregulating or increasing the level or quantity of target expression or presence either within a cell (i.e. intracellular) or on the cell-surface of cancer cells (i.e. extracellular), to specified anti-target agents, drugs targeted against specific cell-surface, or intracellular targets newly identified by the invention methods herein. In other words, the invention priming or contacting step makes cancer cells more susceptible or amenable to treatment or eradication, by specific anti-target agents or drugs that are directed to the newly identified invention priming-eligible targets provided herein.

As used herein the phrase “priming agent” refers to any molecule that is able to epigenetically sensitize cells (e.g., cancer cells), e.g., upon contact with the cells, by controllably upregulating or increasing the level or quantity of a particular target's intracellular or extracellular expression, to specified anti-target agents or drugs targeted against specific intracellular or extracellular targets (e.g., priming targets) newly identified by the invention methods herein. In addition to upregulating and increasing the presence of a particular target, in certain embodiments, the priming agent, either as a single agent or a combination of agents, can also downregulate, decrease or suppress the presence of certain proteins or targets, e.g., PD-L1 and the like. Priming agents with such dual function are referred to as “priming-tuning agents.” The term “priming agent” is used herein to specifically include priming-tuning agents.

As used herein the phrase “upregulating or increasing the level or quantity of target expression or presence either within a cell or on the cell-surface of cancer cells” refers to increasing the level or quantity of intracellular or extracellular target expression or presence by about 2-fold or more. In other embodiments, the increase is in the range of at least about 2-fold up to about 1,000-fold greater than the level or quantity of intracellular or extracellular target expression or presence in the absence of contact with the respective priming agent or agents. In yet other embodiments, the increase is at least about 3-fold, about 4-fold, about 5-fold, about 10-fold, about 20-fold, about 30-fold, about 40-fold, about 50-fold, about 75-fold, about 100-fold, about 200-fold, about 300-fold, about 400-fold, about 500-fold, about 600-fold, about 700-fold, about 800-fold, about 900-fold, about 1,000-fold or more greater than the level or quantity of target intracellular or extracellular expression or presence in the absence of contact with the respective priming agent or agents.

As used herein, the phrase “downregulate, decrease or suppress the presence of certain proteins or targets” refers to decreasing the level or quantity of intracellular or extracellular target expression or presence by about 2-fold or more. In other embodiments, the decrease is in the range of at least about 2-fold up to about 1,000-fold lower than the level or quantity of intracellular or extracellular target expression or presence in the absence of contact with the respective priming agent or agents. In yet other embodiments, the decrease is at least about 3-fold, about 4-fold, about 5-fold, about 10-fold, about 20-fold, about 30-fold, about 40-fold, about 50-fold, about 75-fold, about 100-fold, about 200-fold, about 300-fold, about 400-fold, about 500-fold, about 600-fold, about 700-fold, about 800-fold, about 900-fold, about 1,000-fold or more lower than the level or quantity of intracellular or extracellular target expression or presence in the absence of contact with the respective priming agent or agents.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “treatment,” “treat,” and “treating” refer to reversing, alleviating, delaying the onset of, or inhibiting the progress of a “pathological condition” (i.e., a disease, disorder, or condition, or one or more signs or symptoms thereof) described herein. In some embodiments, “treatment,” “treat,” and “treating” require that signs or symptoms of the disease disorder or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

As used herein, the terms “condition,” “disease,” and “disorder” are used interchangeably.

An “effective amount” of a therapeutic compound used herein refers to an amount sufficient to elicit the desired biological response, i.e., treating the condition. As will be appreciated by those of ordinary skill in this art, the effective amount of a therapeutic compound employed herein may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound used herein, e.g., for treating cancer, is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound used herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: i. the pathological proliferation of normally quiescent or non-quiescent cells; ii. the pathological migration of cells from their normal location (e.g., metastasis of neoplastic cells); iii. the pathological expression of proteolytic enzymes such as the matrix metalloproteinases (e.g., collagenases, gelatinases, and elastases); or iv. the pathological angiogenesis as in proliferative retinopathy and tumor metastasis. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be benign or malignant, depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (i.e. anaplasia) and has characteristically rapid growth accompanied by progressive infiltration, invasion, and destruction of the surrounding tissue. Furthermore, a malignant neoplasm generally has the capacity to metastasize to distant sites.

As used herein, the term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endothelio sarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In certain embodiments of the invention methods provided herein, AML cells are specifically assayed for priming targets and are specifically treated by the invention methods herein.

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases).

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, bone marrow biopsy, bone marrow aspirate, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as transgenic oocyte, sperm cell, blastocyst, embryo, fetus, donor cell, or cell nucleus.

Identifying Priming Agents:

The priming agent for use herein can be identified by a first high-throughput assaying (or “screening”) of single and combination drugs on cancer patients, e.g., Acute Myeloid Leukemia (AML) patients and the like, samples and/or cell lines to identify agents or drug treatments that selectively increase (or prime) expression of a target surface marker (also referred to herein as a “priming eligible target” or “druggable target”). Second, a subsequent high-throughput counter screening on healthy human samples and/or cell lines is conducted to identify which of the agents or drug treatments that were identified in the first cancer patient screening, do not prime (increase or upregulate) the expression of the respective target surface markers (priming eligible targets) on “normal samples.” The agents that upregulate or increase the level or quantity of a particular target's intracellular or extracellular expression for a particular proliferative disease and/or cancer patient population, and not for the respective healthy human normal samples, are provided herein as “priming agents” for using in the invention priming methods and compositions. In addition to upregulating and increasing the presence of a particular target, in certain embodiments, the priming agent, either as a single agent or a combination of agents, can also downregulate, decrease or suppress the presence of certain proteins or targets, such as suppressing expression of at least one surface marker antagonizing T-cells e.g., PD-L1 and the like. Such priming agents are referred to as “priming-tuning” agents.

As used herein, the phrase “normal samples” refers to samples from otherwise healthy individuals who are not known to have the respective proliferative disease or cancer being assayed.

In some embodiments of the invention methods, exemplary priming agents for use herein include, e.g., either single agents, or composition(s) comprising any combination of the following agents, selected from Dexamethasone; Tretinoin; Calcitriol; Panobinostat; Dexamethasone and Tretinoin; Dexamethasone and Panobinostat; Tretinoin and Panobinostat; Dexamethasone and Tretinoin and Calcitriol; and/or Dexamethasone and Tretinoin and Panobinostat, and the like.

### Identifying Priming Targets:

Provided herein are methods for identifying druggable therapeutic targets (also referred to herein as priming-eligible targets), comprising:

a. contacting a test-cancerous-cell and a non-cancerous-control-cell with one or more priming agents;

b. assaying marker expression on both the test-cancerous-cell and the non-cancerous-control-cell in response to the priming agent; and

c. identifying as a candidate therapeutic target (“priming-eligible target) any markers that have increased expression in the test-cancerous-cell compared to both the non-cancerous-control-cell and a control-cancerous cell.

As used herein, the phrase “test-cancerous-cell” refers to a cell or cell-line from a patient known to have cancer or another proliferative disease.

As used herein, the phrase “non-cancerous-control-cell” refers to a cell or cell-line obtained from a healthy individual who is not known to have cancer or another proliferative disease.

As used herein, the phrase “control-cancerous cell” refers to a cell or cell-line from a patient known to have cancer or another proliferative disease not contacted by a priming agent.

In another embodiment, the invention method contemplates further assaying surface marker expression on a control-cancerous-cell that was not contacted with priming agents, and identifying as a candidate therapeutic agent (“prime-eligible target”) any markers that have increased expression in the test-cancerous cell upon contact with one or more priming agents as compared to both the non-cancerous-control-cell and the control-cancerous-cell. As used herein, the “control-cancerous-cell” is the same cell type as the test-cancerous-cell, but is not contacted with the particular priming agents being assayed. The marker expression can be either cell-surface or intracellular marker expression.

Thus, also provided herein, is a method for identifying druggable cancer targets (such as priming-eligible targets as set forth herein), comprising:

a. contacting a test-cancerous-cell, and a non-cancerous-control-cell, with one or more priming agents; and

b. assaying surface marker expression on both the test-cancerous-cell and the non-cancerous-control-cell in response to the priming agent, and further assaying surface marker expression on a control-cancerous-cell that was not contacted with priming agents;

wherein increased surface marker expression of a particular target on the test-cancerous-cell compared to both the control-cancerous-cell and the non-cancerous-control-cell identifies a druggable target.

The invention methods are useful to identify Priming Targets that are either cell-surface marker (e.g., various clusters of differentiation (CD)) or intracellular markers for use in treating cancer. In one particular embodiment, AML patient samples or cell lines are used as the “test-cancerous-cell” and are treated or contacted with known priming agents described herein (e.g., dexamethasone, ATRA, and the like) and then assayed for changes in the surfaceome that only occur when AML samples are treated but not when healthy samples corresponding to the “non-cancerous-control-cell” are treated (e.g., contacted with the particular priming agent(s)). Exemplary changes in the surface markers expression can include: i. an increase or decrease in expression of a target protein or combination of target proteins; ii. an increase or decrease in expression of different isoforms of a target protein or combination of target proteins; iii. a change in the glycosylation state of different isoforms of a target protein or combination of target proteins; iv. changes in other post-translational modification of a target protein or combination of target proteins; and the like.

As used herein, the term “surfaceome” refers to the data set generated using whole transcriptome-based analyses filtered for plasma membrane protein expression.

Various methods for assaying for the changes in priming target/surface marker expression are well known in the art. Exemplary assays for detecting changes include, for example: i. use of multicolor flow cytometry to assay for changes in the surfaceome; ii. use of mass cytometry to assay for changes in the surfaceome; iii. use of RNA-seq to assay for changes in surfaceome; and the like.

In particular embodiments of the invention methods, the target is selected from the group consisting of: CD126, CD44, CD24, and CD15.

In a particular embodiment, the target is CD126. In this particular CD126 embodiment, the priming agents include, e.g., either single agents, or composition comprising any combination of the following agents, selected from Dexamethasone; Tretinoin; Calcitriol; Panobinostat; Dexamethasone and Tretinoin; Dexamethasone and Panobinostat; Tretinoin and Panobinostat; Dexamethasone and Tretinoin and Calcitriol; and/or Dexamethasone and Tretinoin and Panobinostat, and the like. In a particular embodiment, the priming agent is a combination of Dexamethasone and Tretinoin.

In another particular embodiment, the target is CD44. In this particular CD44 embodiment, the priming agents include, e.g., either single agents, or composition comprising any combination of the following agents, selected from Dexamethasone; Tretinoin; Calcitriol; Panobinostat; Dexamethasone and Tretinoin; Dexamethasone and Panobinostat; Tretinoin and Panobinostat; Dexamethasone and Tretinoin and Calcitriol; and/or Dexamethasone and Tretinoin and Panobinostat, and the like. In a particular embodiment, the priming agent is a combination of Calcitriol and Panobinostat.

In yet another particular embodiment, the target is CD24. In this particular CD24 embodiment, the priming agents include, e.g., either single agents, or composition comprising any combination of the following agents, selected from Dexamethasone; Tretinoin; Calcitriol; Panobinostat; Dexamethasone and Tretinoin; Dexamethasone and Panobinostat; Tretinoin and Panobinostat; Dexamethasone and Tretinoin and Calcitriol; and/or Dexamethasone and Tretinoin and Panobinostat, and the like. In a particular embodiment, the priming agent is a combination of Dexamethasone and Tretinoin.

In other embodiments of the invention methods, the target is selected from the group consisting of: CD10, CD100, CD101, CD102, CD103, CD104, CD105, CD106, CD107a, CD107b, CD108, CD109, CD111, CD112, CD113, CD116, CD117, CD118, CD119, CD11a, CD11b, CD11c, CD120a, CD121a, CD121b, CD122, CD123, CD124, CD125, CD127, CD13, CD130, CD131, CD132, CD133, CD135, CD136, CD137, CD14, CD140a, CD140b, CD141, CD142, CD143, CD144, CD146, CD147, CD148, CD150, CD154, CD155, CD156a, CD156b, CD156c, CD157, CD158b2, CD158e, CD158f1, CD158h, CD158i, CD160, CD162, CD163, CD164, CD166, CD167b, CD169, CD16a, CD16b, CD170, CD171, CD172a, CD172b, CD172g, CD18, CD180, CD181, CD183, CD185, CD19, CD194, CD197, CD1b, CD1c, CD1d, CD2, CD20, CD200, CD201, CD202b, CD203c, CD205, CD206, CD208, CD21, CD213a1, CD213a2, CD217, CD218a, CD22, CD220, CD221, CD222, CD226, CD228, CD229, CD23, CD230, CD232, CD239, CD243, CD244, CD25, CD26, CD265, CD269, CD27, CD272, CD273, CD274, CD275, CD276, CD279, CD28, CD280, CD281, CD282, CD283, CD284, CD289, CD29, CD294, CD295, CD298, CD30, CD302, CD304, CD305, CD307, CD31, CD312, CD315, CD316, CD317, CD318, CD319, CD32, CD321, CD322, CD324, CD325, CD326, CD327, CD328, CD33, CD331, CD332, CD333, CD334, CD337, CD339, CD34, CD340, CD344, CD35, CD3d, CD3e, CD3g, CD4, CD40, CD41, CD42d, CD44, CD45, CD46, CD47, CD48, CD49a, CD49b, CD49c, CD49d, CD49e, CD49f, CD5, CD50, CD51, CD54, CD55, CD56, CD58, CD59, CD6, CD61, CD62L, CD62P, CD63, CD64, CD66a, CD66b, CD66c, CD68, CD7, CD71, CD73, CD74, CD79a, CD79b, CD8a, CD8b, CD80, CD83, CD84, CD85f, CD85i, CD85j, CD86, CD87, CD89, CD90, CD91, CD92, CD95, CD96, CD97, CD98, CDw210a, CDw210b, and the like.

### Priming-Tuning:

As used herein, the phrase “priming-tuning,” or grammatical variations thereof, refers to treating cancers with one or a combination of priming agent(s) that function to both increase expression of a target surface marker (priming target) while simultaneously suppressing expression of an undesired target surface protein (such as PD-L1, and the like). This approach allows for maximal efficacy of immunotherapeutic agents by both increasing the expression of the target surface protein (i.e., priming target) on cancerous cells while suppressing the cancer cells ability to escape therapeutic agents.

Priming agents as described above are used to boost the expression of a priming target, e.g., a cancer surface marker. For particular embodiments, such as immunotherapies like CAR-T, ADCC mediated mABs, or BiTEs, it is also desirable to suppress expression of surface markers that antagonize T-cells such as, for example, Programmed Death-Ligand 1 (PD-L1) and the like. Cells targeted by a CAR-T can avoid targeted killing (immunoevasion) by expressing PD-L1 which suppresses the specific cytotoxic activity of the CAR-T. In accordance with one embodiment of the present invention, a single priming agent, such as Dexamethasone, Tretinoin, and the like, that functions to both suppress expression of PD-L1 (or surface markers that antagonize T-cells), while simultaneously increasing expression of a primed target on target cells can be used to prevent immunoevasion. As discussed before, such priming agents are referred to as “priming-tuning agents.”

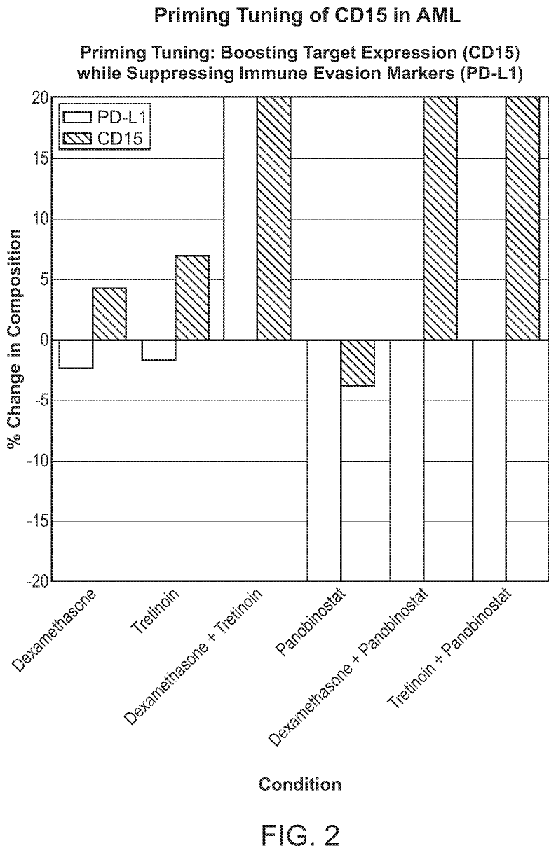

In one embodiment, an approach to mitigate the PD-L1 mediated suppression of CAR-Ts is to use an agent that functions as a checkpoint inhibitor that blocks the activity of PD-L1, such as Panobinostat, in combination with other priming agents for “priming tuning” (see, e.g., FIG. 2). Exemplary combinations for priming tuning provided herein, include Dexamethasone and Panobinostat; Tretinoin and Panobinostat, and the like (FIG. 2).

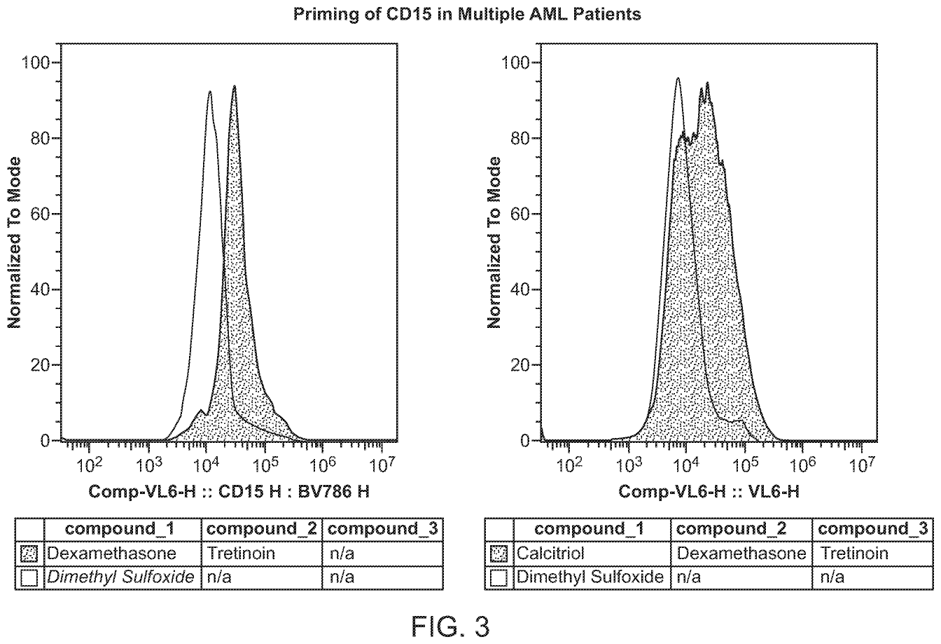

FIG. 3 demonstrates how the invention methods and platform can be used for Priming Tuning. In this embodiment, CD15 is the priming target (i.e. cancer surface marker) that increases in response to several different priming agents on both the right side and left side of the plot. However, several priming agents on the right side of the plot like Brequinar Sodium increase CD15 expression, but also increase PD-L1 expression, which may, in some embodiments, limit their utility as priming agents for CAR-Ts since primed cells may now evade the immune system via PD-L1 mediated checkpoint activation. In contrast, the combination of Dexamethasone and Panobinostat causes simultaneously the priming of CD15 (i.e., increased CD15 surface marker expression) and reduction of PD-L1 expression, thus reducing the cancer's ability to evade targeted immunotherapy.

In other embodiments, ADC agents in contrast to CAR-T or mAB/ADCC mediated antibodies/Bispecific therapies do not require priming tuning to benefit from the priming of the primary surface marker target. ADCs are contemplated herein to maintain their cytotoxic effect independent of PD-L1 mediated inhibition of immune response as their toxicity primarily relies on delivery of a toxic payload to target cells that selectively endocytose the ADCC mediated antibody. That said, in other embodiments, priming tuning may still provide some benefit to therapies that do not immediately rely on T-cell based targeted killing. For example, while ADCs induce apoptosis on target cells without the need for endogenous T-cells, the apoptotic environment is immunogenic and induces an antitumor immune response. In fact, ADCs with checkpoint inhibitors have been shown to have synergistic anti-tumor effects and may even induce immunologic memory (see, e.g., Rios-doria J et al, 2017. Antibody-Drug Conjugates Bearing Pyrrolobenzodiazepine or Tubulysin Payloads Are Immunomodulatory and Synergize with Multiple Immunotherapies. Cancer Research).

### Priming Conditions for Different Therapeutic Modalities:

For the specific embodiment where CD15 is being primed, as shown In FIG. 3, priming conditions that upregulate CD15 while suppressing PD-L1 are contemplated herein, in certain embodiments, to be ideal for anti-CD15 CAR-T, mAB/ADCC mediated antibodies, BiTE antibodies, or ADC therapy. For example, FIG. 3 indicates that the following combinations: Dexamethasone and Panobinostat; Tretinoin and Panobinostat; Panobinostat and Vismodegib; and Calcitriol and Dexamethasone and Tretinoin, are particularly useful for priming CD15 as a primed target, while suppressing PD-L1, for subsequent or co-administration of an anti-CD15 CAR-T, mAB/ADCC mediated antibody, BiTE antibody, or ADC cancer therapeutic agent, or any other anti-CD15 therapeutic agent.

In other embodiments, as set forth in FIG. 3, priming conditions that upregulate CD15 while also increasing PD-L1, while potentially not appropriate for CAR-T/mABs, are believed to be useful for the ADC modality of cancer therapy. For example, the combinations of Brequinar Sodium and Olaparib; Sorafenib and Tretinoin; Brequinar Sodium and Dexamethasone and Tretinoin; Dexamethasone and Tretinoin, are contemplated herein as particularly useful with anti-CD15 ADC therapy.

### CD15 as Priming Target:

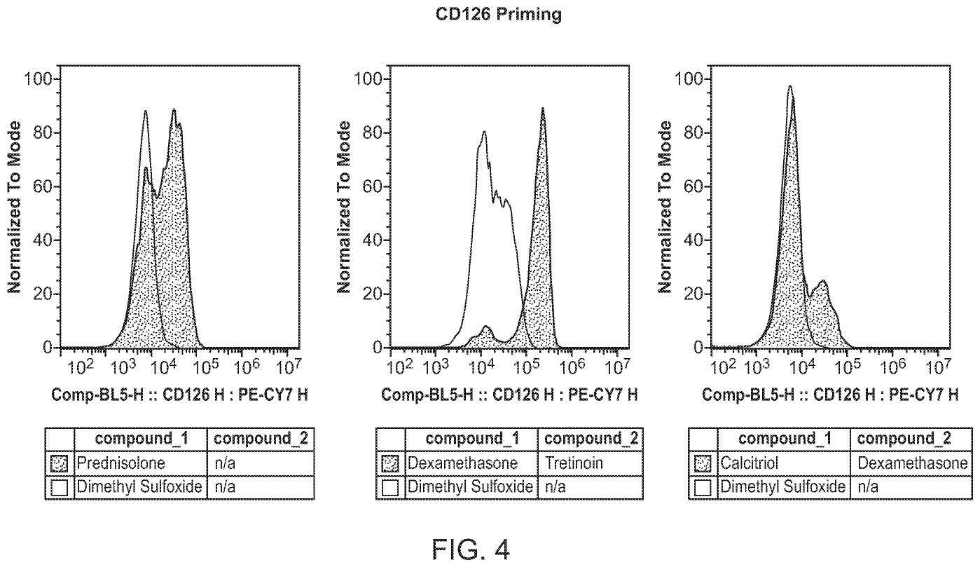

In accordance with the present invention, it has been found that CD15 has several qualities that make it a robust target for use in the priming therapy invention methods provided herein. For example, FIGS. 3 and 4 demonstrate that CD15 expression increases in a subset of AML patient samples that have been treated with a particular priming agent. CD15 does not prime in healthy bone marrow samples. It is well-known in the art that baseline expression of CD15 in healthy tissue is very low and largely restricted to the myeloid compartment. This indicates that even without priming, toxicity in healthy tissue treated with an Anti CD15 therapy would be low (at least in regard to on-target binding of CD15 to noncancerous cells).

In accordance with a particular embodiment of the present invention, the ideal targeting modality for CD15 is likely a CAR-T or Bispecific Antibody (CD15×CD3) because CD15 is not an internalized receptor and is not known to rapidly internalize. As such, an anti-CD15 CAR-T or Bispecific antibody would just need to seek out and find primed cells expressing CD15.

In some embodiments, although ADCs against CD15 in comparison may be suboptimal since CD15 is not known to rapidly internalize when binding an extracellular ligand, CD15 may still be amenable to an ADC therapeutic approach since cell surface proteins still have turnover that involves endocytosis and as such we would expect the ADC to be internalized and deliver their toxic payload to targeted cells.

### Modalities:

For the invention therapeutic targets identified and/or provided herein, any therapeutic agent that is known to specifically bind to the respective target via any modality is contemplated for use in the invention therapeutic methods. Exemplary therapeutic agents for use herein include, e.g., ADCs, CAR-T, mAB/ADCC mediated antibodies, BiTE antibodies, and the like; so long as they are therapeutically active against a specified therapeutic target identified herein.

### ADCs:

For example, ADCs are provided herein for use in the invention therapeutic methods. As set forth in U.S. Pat. No. 8,603,483 (incorporated herein by reference in its entirety), invention ADCs are represented by the Formula:

[C-L]ₘ-A,

where A is a specific priming target antibody (such as an anti-CD126, anti-CD44, anti-CD24, anti-CD15 and the like) wherein said antibody is capable of being internalized by the cell expressing the respective CD subunit (e.g., CD126, CD44, CD24, CD15, and the like); C is a cytotoxin with a half maximal inhibitory concentration (IC50) of, for example, 10⁻⁹ M or less; and L is linking group which binds the antibody and cytotoxin and further comprises a bond cleavable by components of the intracellular environment; and m represents the average number of cytotoxin molecules linked to the antibody and is an integer from 1-10, or in other embodiments, from 1-5. In another embodiment, the drug:antibody ratio is from about 2-4. In a particular embodiment, the drug:antibody ratio is 2. In another embodiment, the drug:antibody ratio is 3. In yet another embodiment, the drug:antibody ratio is 4. The cytotoxin may be selected from any of the toxins well known in the art. For example, the cytotoxin can be selected from any one or any combination of the group consisting of maytansinoids (e.g., DM1 [CAS Reg. No. 139504-50-0], DM3, DM4 or the like), calicheamicins, doxorubicins, duocarmycins, pyrrolobenzodiazepines (PBDs), Topotecan, Bleomycin A2, Mitomycin C, Dactinomycin analogs, Shiga-like toxin, epothilones, Discodermolide, eleuthrobins, dolastatins, cryptophycins, camptothecins, Rhizoxin (CAS reg. no. 90996546), or taxane derivatives and such other compounds that exhibit, in some embodiments, IC50 or half maximal growth inhibiton (GI50) of on tumor cell growth at 10⁻⁹ M or less.

For an ADC directed to the CD126 priming target provided herein, exemplary anti-CD126 antibodies include Tocilizumab, Sarilumab, Siltuximab, Elsilimab, Sirkumab, and the like. For an ADC directed to the CD44 priming target provided herein, exemplary anti-CD44 antibodies include Bivatuzumab, RG7536 (or R05429083; set forth in Oncotarget. 2016 Nov. 29; 7(48): 80046-80058; incorporated herein by reference in its entirety), and the like. For an ADC directed to the CD24 priming target provided herein, exemplary anti-CD24 antibodies include CD24 Fc (Oncolmmune). For an ADC directed to the CD15 priming target provided herein, exemplary anti-CD15 antibodies include MDX 22 and MDX 11.

It is known in the preparation of conjugates of two substances, of which at least one comprises a protein or a polypeptide, to use bifunctional agents in order to couple the components of the conjugate covalently, amino groups in the conjugated molecules normally being utilized for the conjugating reaction (“Linkers” or “Bifunctional Coupling Reagents”). Bifunctional coupling agents include N-succinimidyl-(2-pyridyldithio)propionate (SPDP), succinimidyl-4-(N-maleimidomethyl)cyclohexane-1-carboxylate, iminothiolane (IT), bifunctional derivatives of imidoesters such as dimethyl adipimidate.HCl, active esters such as disuccinimidyl suberate, aldehyes such as glutaraldehyde, bis-azido compounds suc has bis(p-axidobenzoyl)hexanediamine, bis-diazonium derivatives such as bis-(p-diazoniumbenzoyl)-ethylenediamine), diisocyanates (such as tolyene 2,6-diisocyanate), and bis active fluorine compounds such as 1,5-difluoro-2,4-dinitrobenzene). SPDP is among the most frequently used reagent for this purpose and many other N-succinimidyl-(2-pyridyldithio)-, N-succinimidyl-(5-nitro-2-pyridyldithio)- or N-succinimidyl-(4-pyridyldithio)-short chain alkane acids have proved useful.

Linkers comprising intracellularly cleavable bonds include acid-labile linkages such as cis-aconityl linkages, esters, acid-sensistive hydrazone linkages, lysosomally degradable peptide linkers, hydrolase cleavable linkers, peptidase or protease specific linkers, and disulfide (sulphydryl) linkers (see Dyba, M., et al. 2004 Curr Pharm Design 10:2311-2334 for a review; incorporated herein by reference in its entirety for all purposes). By being capable of more rapid or selective cleavage under intracellular conditions versus the conditions predominating in, for example, the circulation, the linker imparts further specificity and safety to the overall pharmacodynamics of the conjugate. In certain embodiments, disulfide linkages are particularly preferred because of the favorable reduction potential within the cellular compartments as well as inducible redox enzyme activation (Saito, G. et al. Adv. Drug Delivery Rev 2003 55:199-215). In one embodiment of the invention, the bond is between a sulfur atom present in the antibody molecule, e.g. in the side chain of a cysteine residue, and another sulfur atom present in the toxic compound. In another embodiment, the linking moiety consists of one or more atoms or chemical groups.

Conjugates of the antibody molecules of the invention and toxic compound can be formed using any techniques presently known or later developed. For example, the cytotoxic compound can be modified to yield a free amino group and then linked to the antibody molecule via an acid-labile linker, or a photolabile linker. The toxic compound can be condensed with a peptide and subsequently linked to an antibody molecule to produce a peptidase-labile linker. The toxic compound can be treated to yield a primary hydroxyl group, which can be succinylated and linked to an antibody molecule to produce a conjugate that can be cleaved by intracellular esterases to liberate free drug.

In order to create the disulfide linkage between antibody A and the cytotoxin C, preferably, the toxic compound is treated to create a free or protected thiol group and then one or many disulfide or thiol containing toxic compounds are covalently linked to the antibody molecule via disulfide bond(s). The disulfide bond need not be formed directly with a free thiol of the antibody but can be formed by derivatization of any reactive group within the antibody to introduce a site for disulfide exchange, for example, as by coupling a bifunctional linker to free amine groups in the antibody. For example, antibody molecules can be modified with crosslinking reagents such as N-succinimidyl 3-(2-pyridyldithio)propionate (SPDP), 4-succinimidyl-oxycarbonyl-a-methyl a-(2-pyridyldithio)-toluene (SMPT), N-succinimidyl-3-(2-pyridyldithio)-butyrate (SDPB), N-succinimidyl-4-(2-pyridyldithio) pentanoate (SPP), N-succinimidyl-5-(2-pyridyldithio)pentanoate, 2-iminothiolane (IT), or acetylsuccinic anhydride by known methods.

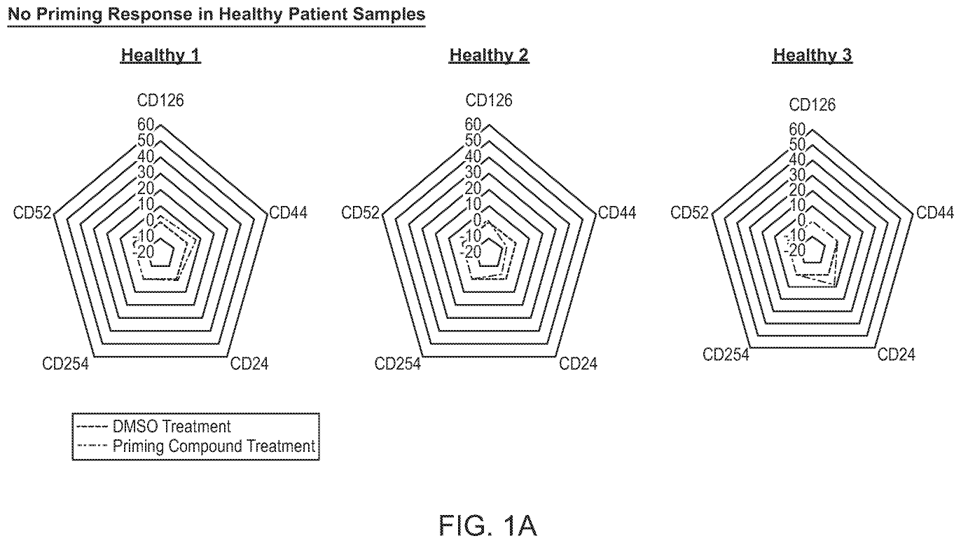

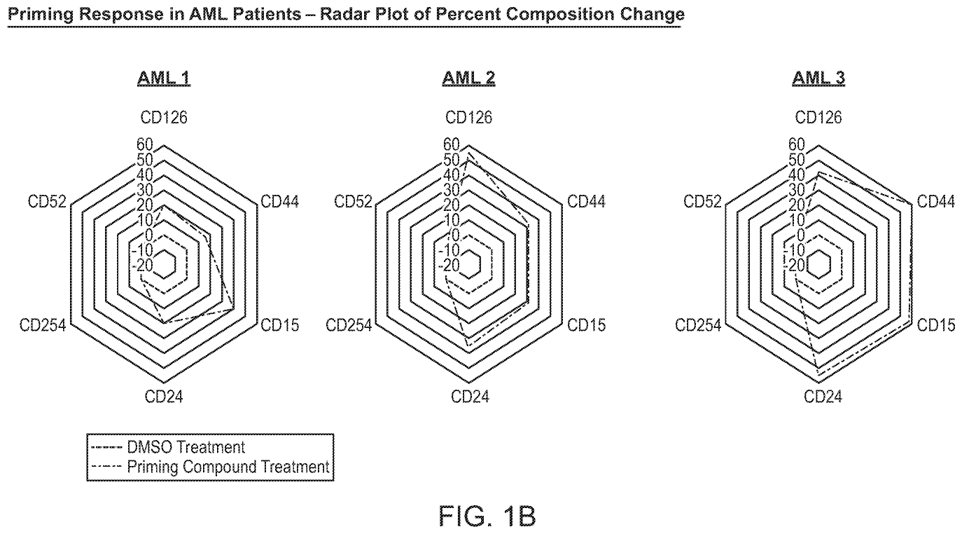

The anti-primed target antibody-maytansinoid conjugates of the invention are prepared by chemically linking an anti-primed target antibody to a maytansinoid molecule without significantly reducing the biological activity of the antibody and providing a maytansinoid, which when released under physiological conditions, retains its cytotoxic potential. Examples of suitable maytansinoids are esters of maytansinol and maytansinol analogues including, but not limited to those having a modified aromatic ring and those having modifications at C-19, C-20, or C-14, or C-15, or C-4,5 deoxy. In certain embodiments, preferred are maytansinol C-3 esters. In other particular embodiments, preferred maytansinoids are derivatives of N-methyl-alanine esters of maytansinol (N2′-deacetyl-maytansine). In other particular embodiments, preferred conjugates comprise a disulfide linkage, which when cleaved by reduction, releases a corresponding maytansinoid bearing a free thiol. Thiol containing maytansinoids of the preferred type are shown in FIG. 1 of U.S. Pat. No. 8,603,483 as: DM1, CAS Reg. No. 139504-50-0, DM3, and DM4, which patent is incorporated herein by reference in its entirety.

In a particular embodiment, the linker moiety is a 4-thiopentanoate derived from SPP, or 4-thiopentanoate. The antibody molecule containing free or protected thiol groups thus derived, is then reacted with a disulfide- or thiol-containing toxic compound to produce conjugates. The conjugates can be purified by high-performance liquid chromatography (HPLC) or by gel filtration.

Accordingly, in particular embodiments, ADCs provided herein include: DM1-4-thiopentanoate-anti-CD126, DM1-4-thiopentanoate-anti-Tocilizumab, DM1-4-thiopentanoate-anti-Sarilumab, DM1-4-thiopentanoate-anti-Siltuximab, DM1-4-thiopentanoate-anti-Elsilimab, DM1-4-thiopentanoate-anti-Sirkumab; DM1-4-thiopentanoate-anti-CD44, DM1-4-thiopentanoate-anti-bivatuzumab, DM1-4-thiopentanoate-anti-RG7536; DM1-4-thiopentanoate-anti-CD24, DM1-4-thiopentanoate-anti-CD24 Fc; DM1-4-thiopentanoate-anti-CD15, DM1-4-thiopentanoate-anti-MDX 22, DM1-4-thiopentanoate-anti-MDX 11; and the like.

In other particular embodiments, ADCs provided herein include: DM3-4-thiopentanoate-anti-CD126, DM3-4-thiopentanoate-anti-Tocilizumab, DM3-4-thiopentanoate-anti-Sarilumab, DM3-4-thiopentanoate-anti-Siltuximab, DM3-4-thiopentanoate-anti-Elsilimab, DM3-4-thiopentanoate-anti-Sirkumab; DM3-4-thiopentanoate-anti-CD44, DM3-4-thiopentanoate-anti-bivatuzumab, DM3-4-thiopentanoate-anti-RG7536; DM3-4-thiopentanoate-anti-CD24, DM3-4-thiopentanoate-anti-CD24 Fc; DM3-4-thiopentanoate-anti-CD15, DM3-4-thiopentanoate-anti-MDX 22, DM3-4-thiopentanoate-anti-MDX 11; and the like.

In yet other particular embodiments, ADCs provided herein include: DM4-4-thiopentanoate-anti-CD126, DM4-4-thiopentanoate-anti-Tocilizumab, DM4-4-thiopentanoate-anti-Sarilumab, DM4-4-thiopentanoate-anti-Siltuximab, DM4-4-thiopentanoate-anti-Elsilimab, DM4-4-thiopentanoate-anti-Sirkumab; DM4-4-thiopentanoate-anti-CD44, DM4-4-thiopentanoate-anti-bivatuzumab, DM4-4-thiopentanoate-anti-RG7536; DM4-4-thiopentanoate-anti-CD24, DM4-4-thiopentanoate-anti-CD24 Fc; DM4-4-thiopentanoate-anti-CD15, DM4-4-thiopentanoate-anti-MDX 22, DM4-4-thiopentanoate-anti-MDX 11; and the like.

### Methods of Preparation of Maytansinol ADCs:

The starting compound, maytansinol, as used in the production of compounds DM1, DM3 and DM4 and related activated maytansinoids according to this invention can be prepared from maytansine a natural C-3 ester isolated from natural sources (Kupchan et al., J. Amer. Chem. Soc. 97, 5294(1975)) by reductive cleavage. The reagent lithium trimethoxyaluminum hydride in tetrahydrofuran at −40° C. is particularly useful for this step. Other natural maytansinoid esters may also be advantageously produced by cultivating microorganisms, which belongs to the genus Nocardia (U.S. Pat. No. 4,151,042) or Actinosynnema spp. that have been engineered to produce maytansinol, maytanacine or C-3 maytansinol esters such as maytansinol propionate in the culture broth and extracting the compounds from the culture broth for further purification. As set forth above, there are many linking groups known in the art for making antibody maytansinoid conjugates, including, for example disulfide groups, thioether groups, acid labile groups, photolabile groups, peptidase labile peptide linkers, or esters which may be acid labile or esterase cleavable.

As taught in U.S. Pat. No. 5,208,020; esterification of maytansinol or an analogue with the carboxylic acids containing a methyldithio group or other protected thio group, including, for example, N-methyl-N-[3-(methyldithio)-1-oxopropyl]-L-alanine produce the corresponding disulfide-containing maytansinoids. In the case where two diastereomeric products containing the D- and L-acyl side chains result, the diastereomeric maytansinoid esters are readily separated by methods known in the art and the less desirable D-alanyl analog isomer product reduced to recover maytansinol as taught in WO03096782. Reductive cleavage of the disulfide group with dithiothreitol gives the corresponding thiol-containing maytansinoid, which is readily linked via disulfide or thioether linkages to cell binding agents. Thiol-maytansinoids can be purified by HPLC using a C18 column in the reverse phase mode eluting with a gradient of water-acetonitrile.

Conjugation of Activated Antibody to Thiolated Maytansinoid: The preparation of, for example, anti CD15-Maytansinoid conjugates follows the method previously described (Chari et al., Cancer Res. 52: 127-131, 1992 and U.S. Pat. No. 5,208,020; which are incorporated herein by reference in their entirety for all purposes). In this procedure, antibody is modified with bifunctional linker at a ratio of linker to antibody in the range of 5 to 10:1 to introduce dithiopyridyl groups onto the antibody amino acid side chains. The activated antibody is separated from residual linker by G25 gel filtration chromatography. The linker antibody ratio after the purification is less than 5 to 10:1 and typically in the range of 3 to 5:1 and is measured by absorbance at 252 nm and 280 nm. The activated thiol-maytansinoid is added at molar excess to that of the measured linker. Following the conjugation, the mixture is again purified by G25 size exclusion chromatography to yield bulk product.

In the alternative, the anti-primed target antibody maytansinoid conjugate is prepared by essentially a single step of reacting a maytansinoid bearing a reactive ester with anti-integrin antibody not previously chemically activated. The reactive ester of the maytansinoid may be a N-succinimidyl, N-sulfosuccinimidyl, N-phthalimidyl, N-sulfophthalimidyl, 2-nitrophenyl, 4-nitrophenyl, 2,4-dinitrophenyl, 3-sulfonyl-4-nitrophenyl or 3-carboxy-4-nitrophenyl ester. The method is described in publication WO2002098883, the contents of which are incorporated herein by reference in their entirety.

### Pharmaceutical Compositions, Kits, and Administration

The present invention provides pharmaceutical compositions comprising an ADC, CAR-T, mAB, or a BiTE antibody, that specifically binds to a target selected from CD126, CD44, CD24, CD15, and the like, as described herein, and optionally a pharmaceutically acceptable excipient. In certain embodiments, as described herein, the pharmaceutical composition of the invention comprises an ADC comprising an antibody selected from the group consisting of an: anti-CD126, anti-CD44, anti-CD24, and an anti-CD15 antibody; a linker, such as 4-thiopentanoate or the like; and a suitable toxin, such as maytansinoid, or the like; and optionally a pharmaceutically acceptable excipient. In certain embodiments, an effective amount in the pharmaceutical composition is a therapeutically effective amount; and in other embodiments, it is a prophylactically effective amount.

Also provided herein are pharmaceutical combination compositions comprising various combinations of priming agents, provided herein, and therapeutically active pharmaceutical compositions provided herein.

Pharmaceutical compositions described herein can be prepared by any method known in the art of pharmacology. In general, such preparatory methods include the steps of bringing the respective compound (i.e., the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (weight by weight) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used. Pharmaceutically acceptable compositions of this invention may also be administered by nasal aerosol or inhalation.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the therapeutic compounds provided herein, such as the invention ADCs may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration (FDA) as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, deoxyribonucleic acids (DNA), ribonucleic acids (RNA), nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease (e.g., cancer (e.g., leukemia, melanoma, and multiple myeloma), benign neoplasm, angiogenesis, inflammatory disease, autoinflammatory disease, or autoimmune disease). The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one unit dosage form.

Also provided herein are kits comprising various combinations of priming agents, provided herein, and therapeutically active pharmaceutical compositions provided herein.

Thus, in one aspect, provided are kits including a first container comprising a compound described herein, or a pharmaceutically acceptable salt, solvate, hydrate, tautomer, stereoisomer, and isotopically labeled derivative, or a pharmaceutical composition thereof. In certain embodiments, the kit of the invention includes a first container comprising a compound described herein, or a pharmaceutically acceptable salt thereof, or a pharmaceutical composition thereof. In certain embodiments, the kit of the invention includes a first container comprising a priming agent. In certain embodiments, the kits are useful in preventing and/or treating a proliferative disease in a subject. In certain embodiments, the kits further include instructions for administering the compound, or a pharmaceutically acceptable salt, solvate, hydrate, tautomer, stereoisomer, isotopically and labeled derivative thereof, or a pharmaceutical composition thereof, to a subject to prevent and/or treat a proliferative disease, such as any of the cancers described herein.

### Methods of Treatment and Uses

The present invention also provides methods for the treatment or prevention of a proliferative disease (e.g., cancer, benign neoplasm, angiogenesis, inflammatory disease, autoinflammatory disease, or autoimmune disease) or an infectious disease (e.g., a viral disease) in a subject. Such methods for treating cancer or a proliferative disease in a subject in need thereof, comprise: priming the subject with a priming agent to increase the level of a specified target; and administering an effective amount of a therapeutic agent that binds to the specified target.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

The proliferative disease or cancer to be treated or prevented using the compounds provided herein will typically be associated with, or otherwise correlated with, increased expression of the specific invention cancer targets corresponding to CD126, CD44, CD24, CD15, or the like. The increased expression of CD126, CD44, CD24, CD15, or the like, is controlled in accordance with the invention methods by the respective priming agent or priming agent combinations used herein to controllably increase the expression of a specified target. In certain other embodiments, CD126 is overexpressed by selected priming agents, and the surface marker expression of CD126 on a cancer cell surface is elevated, such that the cancer cell is rendered more susceptible to therapeutic treatment with an agent that specifically binds to CD126, e.g., anti-CD126 ADC, mAB, T-Cell Receptor (TCR)-engineered T-Cell, or CAR-T cell, or the like. These anti-CD126 compounds, and pharmaceutically acceptable salts, solvates, hydrates, tautomers, stereoisomers, isotopically labeled derivatives, and compositions thereof, are contemplated herein to specifically bind to CD126-overexpressing cells to inhibit cell proliferation and be useful in treating and/or preventing proliferative diseases, such as cancer.

In other embodiments, CD44 is overexpressed by selected priming agents, and the surface marker expression of CD44 on a cancer cell surface is elevated, such that the cancer cell is rendered more susceptible to therapeutic treatment with an agent that specifically binds to CD44, e.g., anti-CD44 ADC, mAB, TCR, or CAR-T cell, or the like. These anti-CD44 compounds, and pharmaceutically acceptable salts, solvates, hydrates, tautomers, stereoisomers, isotopically labeled derivatives, and compositions thereof, are contemplated herein to specifically bind to CD44-overexpressing cells to inhibit cell proliferation and be useful in treating and/or preventing proliferative diseases, such as cancer.

In certain other embodiments, CD24 is overexpressed by selected priming agents, and the surface marker expression of CD24 on a cancer cell surface is elevated, such that the cancer cell is rendered more susceptible to therapeutic treatment with an agent that specifically binds to CD24, e.g., anti-CD24 ADC, mAB, TCR, or CAR-T cell, or the like. These anti-CD24 compounds, and pharmaceutically acceptable salts, solvates, hydrates, tautomers, stereoisomers, isotopically labeled derivatives, and compositions thereof, are contemplated herein to specifically bind to CD24-overexpressing cells to inhibit cell proliferation and be useful in treating and/or preventing proliferative diseases, such as cancer.

In certain other embodiments, CD15 is overexpressed by selected priming agents, and the surface marker expression of CD15 on a cancer cell surface is elevated, such that the cancer cell is rendered more susceptible to therapeutic treatment with an agent that specifically binds to CD15, e.g., anti-CD15 ADC, mAB, TCR, or CAR-T cell, or the like. These anti-CD15 compounds, and pharmaceutically acceptable salts, solvates, hydrates, tautomers, stereoisomers, isotopically labeled derivatives, and compositions thereof, are contemplated herein to specifically bind to CD15-overexpressing cells to inhibit cell proliferation and be useful in treating and/or preventing proliferative diseases, such as cancer.

In certain embodiments, the proliferative disease to be treated or prevented using the invention methods and/or compounds is cancer. All types of cancers disclosed herein or known in the art are contemplated as being within the scope of the invention. In certain embodiments, the proliferative disease is a hematological malignancy. In certain embodiments, the proliferative disease is a blood cancer. In certain embodiments, the proliferative disease is leukemia. In certain embodiments, the proliferative disease is CLL. In certain embodiments, the proliferative disease is ALL. In certain embodiments, the proliferative disease is T-ALL. In certain embodiments, the proliferative disease is CML. In certain embodiments, the proliferative disease is AML. In certain embodiments, the proliferative disease is lymphoma. In certain embodiments, the proliferative disease is melanoma. In certain embodiments, the proliferative disease is multiple myeloma. In certain embodiments, the proliferative disease is a bone cancer. In certain embodiments, the proliferative disease is osteosarcoma. In some embodiments, the proliferative disease is Ewing's sarcoma. In some embodiments, the proliferative disease is triple-negative breast cancer (TNBC). In some embodiments, the proliferative disease is a brain cancer. In some embodiments, the proliferative disease is neuroblastoma. In some embodiments, the proliferative disease is a lung cancer. In some embodiments, the proliferative disease is small cell lung cancer (SCLC). In some embodiments, the proliferative disease is large cell lung cancer. In some embodiments, the proliferative disease is a benign neoplasm. All types of benign neoplasms, such as, MDS, JMML CMML, Mastocytosis, and the like, disclosed herein or known in the art are contemplated as being within the scope of the invention.

The cell described herein may be an abnormal cell. The cell may be in vitro or in vivo. In certain embodiments, the cell is a proliferative cell. In certain embodiments, the cell is a blood cell. In certain embodiments, the cell is a lymphocyte. In certain embodiments, the cell is a cancer cell. In certain embodiments, the cell is a leukemia cell. In certain embodiments, the cell is a CLL cell. In certain embodiments, the cell is a melanoma cell. In certain embodiments, the cell is a multiple myeloma cell. In certain embodiments, the cell is a benign neoplastic cell. In certain embodiments, the cell is an endothelial cell. In certain embodiments, the cell is an immune cell.

In certain embodiments, the methods described herein comprise the additional step of administering one or more additional pharmaceutical agents in combination with the priming agents and/or anti-primed target therapeutic agents described herein, a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment inhibition of CDK7, CDK12, or CDK13 induced by the inventive compounds or compositions of this invention in the biological sample or subject. In certain embodiments, the additional pharmaceutical agent is Flavopiridol, Triptolide, SNS-032 (BMS-387032), PHA-767491, PHA-793887, BS-181, (S)-CR8, (R)-CR8, or NU6140. In certain embodiments, the additional pharmaceutical agent is an inhibitor of a mitogen-activated protein kinase (MAPK). In certain embodiments, the additional pharmaceutical agent is an inhibitor of a glycogen synthase kinase 3 (GSK3). In certain embodiments, the additional pharmaceutical agent is an inhibitor of an AGC kinase. In certain embodiments, the additional pharmaceutical agent is an inhibitor of a CaM kinase. In certain embodiments, the additional pharmaceutical agent is an inhibitor of a casein kinase 1. In certain embodiments, the additional pharmaceutical agent is an inhibitor of a STE kinase. In certain embodiments, the additional pharmaceutical agent is an inhibitor of a tyrosine kinase. Thus, the combination of the inventive compounds or compositions and the additional pharmaceutical agent(s) may be useful in treating proliferative diseases resistant to a treatment using the additional pharmaceutical agent(s) without the inventive compounds or compositions.

In some embodiments, the one or more additional pharmaceutical agents are independently selected from a topoisomerase inhibitor, a MCL1 inhibitor, a BCL-2 inhibitor, a BCL-xL inhibitor, a BRD4 inhibitor, a CDK9 inhibitor, a Jumonji histone demethylase inhibitor, and a DNA damage inducer. In a more specific aspect of these embodiments, the one or more additional agents is selected from Etoposide, Obatoclax, Navitoclax, JQ1, 4-(((5′-chloro-2′-(((1R,4R)-4-(((R)-1-methoxypropan-2-yl)amino)cyclohexyl-)amino)-[2,4′-bipyridin]-6-yl)amino)methyl)tetrahydro-2H-pyran-4-carbonitr-ile, JIB04 and cisplatin. In still another even more specific aspect of these embodiments, the additional agent is selected from JQ1 and NVP2, and the disease to be treated is leukemia, e.g., acute myelogenous leukemia, myeloblastic leukemia, promyelocytic leukemia, myelomonocytic leukemia, monocytic leukemia, monoblastic leukemia, or megakaryoblastic leukemia.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more.”

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

### EXAMPLES

In accordance with the present invention, it has been demonstrated that the invention methods provided herein can be used to assay and identify primed targets that are novel targets for immunotherapy when used in combination with priming agents. We have identified several priming surface markers (e.g. CD126, CD24, CD44, CD15) that are candidate targets for developing therapeutics, such as, e.g., ADCs, and the like.

### Example 1. Priming can be Used to Specifically Increase Target Surface Markers on Leukemic Cells without Altering Marker Expression on Healthy Cells

12 primary AML patient samples were assayed for priming response using a High-Throughput Phenotypic Screening Platform. Samples were treated with 70 priming compounds (either FDA approved compounds or compounds in clinical development). After a three-day incubation period, samples were immunostained and analyzed on a flow cytometer to assess if any surface markers were upregulated in response to any of the priming agents. This process was repeated on bone marrow aspirate and peripheral blood from 3 healthy donors.

Using the invention methods provided herein, several surface markers were identified that leukemic cells upregulate in response to treatment or contact with particular priming agents. Of the set of 10 surface markers assayed for potential priming, CD15, CD126, CD44, and CD24 were consistently upregulated in response to specific priming agents in a subset of patients (FIG. 1B).

In comparison, healthy cells showed little to no change in these surface markers when exposed to the same priming agents. (FIG. 1A).

### Example 2. Specific AML Patient Subsets Respond to Surface Marker Priming

Of the 12 AML patient samples assayed, 3 samples upregulated target surface markers when treated with priming drugs (FIG. 1B). All responsive samples had monoblast populations, fitting either the M4 or M5 classification of AML and only 1 of the M4/M5 samples did not respond to the priming agent.

Priming in accordance with the present invention is believed to specifically target these types AML subtypes, which is particularly significant since M4/M5s have poor prognosis compared to the other major subtypes of adult AML. The specificity of the responses obtained in accordance with the present invention, indicates opportunities for identifying further predictive biomarkers for priming therapy.

### Example 3. Combinations of Priming Agents Optimally Upregulate Different Target Surface Markers

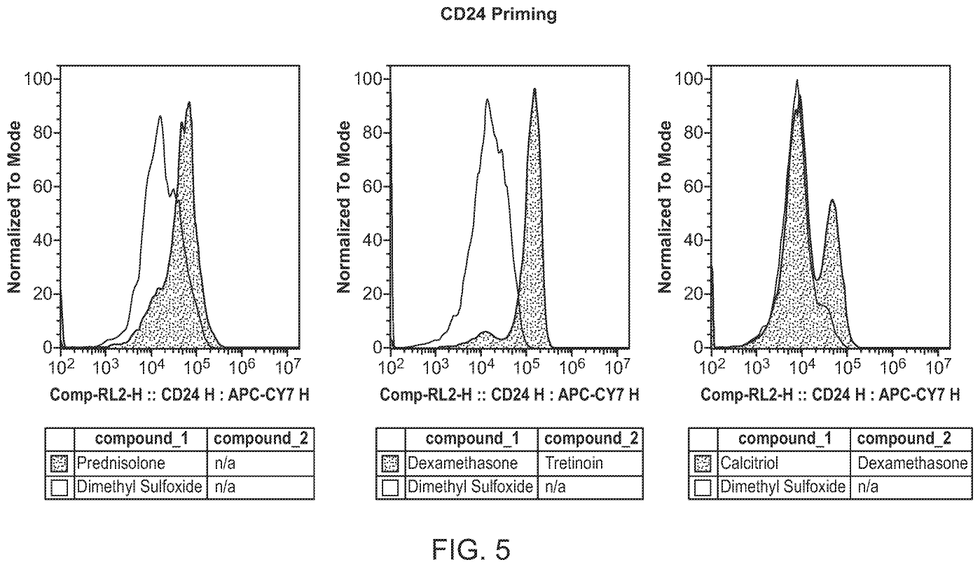

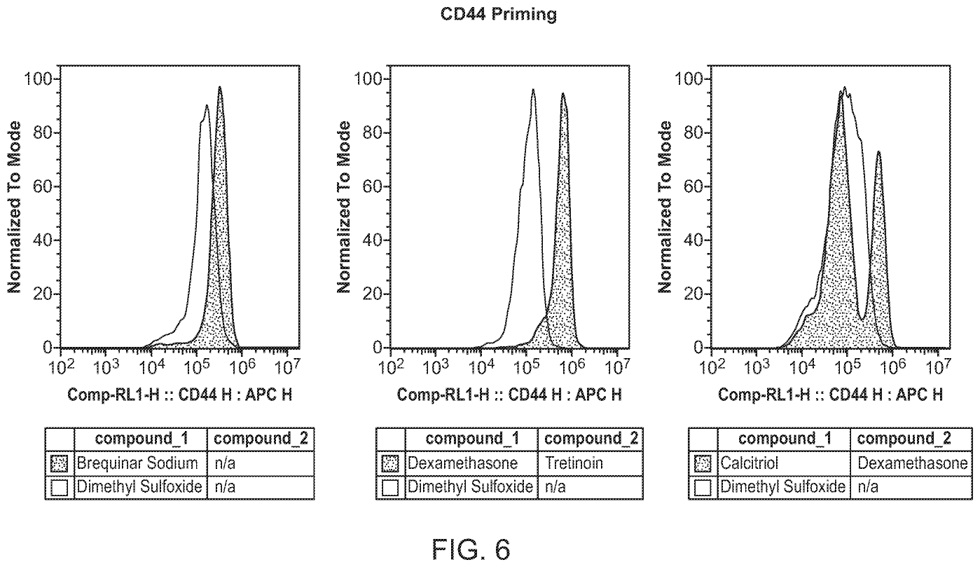

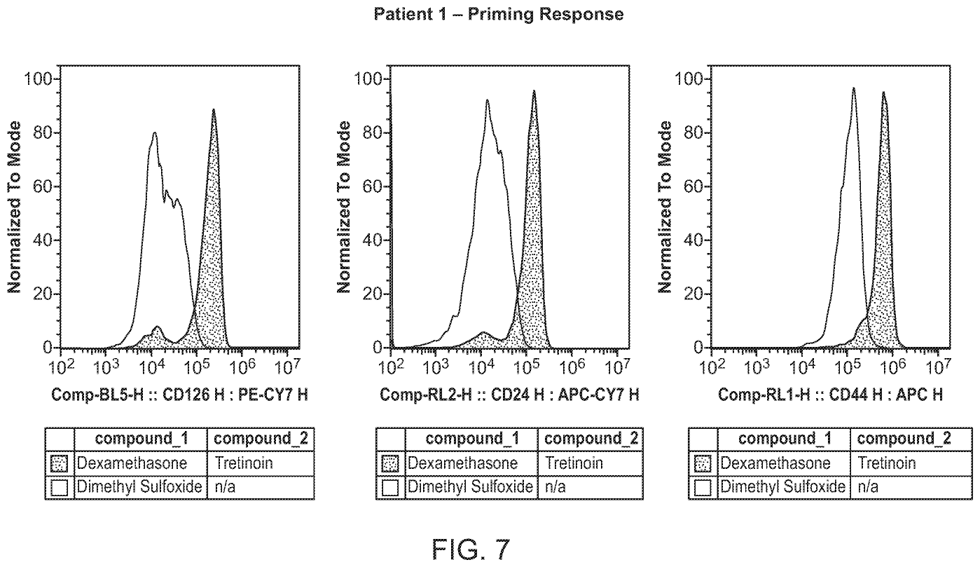

Even when priming agents used as single agents show suboptimal upregulation of surface markers, the high throughput screening platform can overcome this priming blockade by exploring the combination space of priming agents. For example, priming assays demonstrated that for AML Patient 3, the combination of two FDA approved drugs, Dexamethasone and Tretinoin strongly upregulated expression of CD126 and CD24, while either alone had little effect on surface marker expression. Likewise, for AML Patient 1, the combination of Dexamethasone and Tretinoin strongly upregulated expression or the presence of CD126, CD24 and CD44 as set forth in FIGS. 4-7.

### Clinical Validation of the Ex Vivo Assay Predictions

The ex vivo assay used with the invention methods has been found to accurately predict surface marker upregulation clinically. For example, it has been found that a subset of MDS and AML cancer patient samples exhibited CD38 upregulation after exposure to SY-1425 in the 72-hour ex vivo assay. The ex vivo detection of CD38 upregulation using the 72-hour ex vivo assay was clinically validated by trial sites, which compared CD38 expression on leukemic blasts before enrollment and at the beginning of cycle two on SY-1425. As a result of this finding that CD38 expression is upregulated after priming with SY-1425, a new trial arm was initiated that combines SY-1424 with Daratumamab, a FDA approved mAB that targets CD38 in multiple myeloma.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-4. (canceled)

5. A kit comprising one or more priming agents selected from any combination of one or more of Dexamethasone, Tretinoin, Calcitrol, and Panobinostat.

6. The kit of claim 5 further comprising a therapeutically active pharmaceutical composition.

7. The kit of claim 6, comprising the priming agent(s) in a first container and the therapeutically active pharmaceutical composition in a second container.

8. The kit of claim 7, wherein the therapeutically active pharmaceutical composition is to treat a proliferative disease.

9. The kit of claim 8, wherein the proliferative disease is cancer.

10. The kit of claim 9, wherein the cancer is a hematological cancer.

11. A method for treating a proliferative disease in a subject in need thereof, comprising:
a. priming the subject with one or more priming agents to increase the level of a specified target; and
b. administering to the subject an effective amount of a therapeutic agent that binds to the specified target.

12. The method of claim 11, wherein the proliferative disease is cancer.

13. The method of claim 12, wherein the cancer is hematopoietic cancer.

14. The method of claim 13, wherein the hematopoietic cancer is leukemia or lymphoma.

15. The method of claim 14, wherein the leukemia is acute lymphocytic leukemia (ALL), acute myelocytic leukemia (AML), chronic myelocytic leukemia (CML), or chronic lymphocytic leukemia (CLL).

16. The method of claim 14, wherein the lymphoma is Hodgkin's lymphoma (HL), non-Hodgkin's lymphoma (NHL), follicular lymphoma, chronic lymphocytic leukemia/small lymphocytic lymphoma (CLL/SLL), mantle cell lymphoma (MCL), marginal zone B-cell lymphoma, primary mediastinal B-cell lymphoma, Burkitt lymphoma, lymphoplasmacytic lymphoma, hairy cell leukemia (HCL), immunoblastic large cell lymphoma, precursor B-lymphoblastic lymphoma, or primary central nervous system (CNS) lymphoma.

17. The method of claim 11, wherein the priming agent is selected from any combination of one or more of Dexamethasone; Tretinoin; Calcitriol; and Panobinostat.

18. The method of claim 17, wherein the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat.

19. The method of claim 11, wherein the target is selected from the group consisting of: CD126, CD44, CD24, and CD15.

20. The method of claim 19, wherein the target is CD126.

21. The method of claim 20, wherein the priming agent is selected from any combination of one or more of Dexamethasone; Tretinoin; Calcitriol; Panobinostat.

22. The method of claim 21, wherein the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat.

23. The method of claim 22, wherein the priming agent is a combination of Dexamethasone and Tretinoin.

24. The method of claim 19, wherein the target is CD44.

25. The method of claim 24, wherein the priming agent is selected from any combination of one or more of the following agents, selected from Dexamethasone; Tretinoin; Calcitriol; and Panobinostat.

26. The method of claim 25, wherein the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat.

27. The method of claim 19, wherein the target is CD24.

28. The method of claim 27, wherein the priming agent is selected from any combination of one or more of Dexamethasone; Tretinoin; Calcitriol; and Panobinostat.

29. The method of claim 28, wherein the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat.

30. The method of claim 29, wherein the priming agent is a combination of Dexamethasone and Tretinoin.

31. The method of claim 19, wherein the target is CD15.

32. The method of claim 31, wherein the priming agent is selected from any combination of one or more of Dexamethasone; Tretinoin; Calcitriol; and Panobinostat.

33. The method of claim 32, wherein the priming agent is selected from the group consisting of Dexamethasone and Tretinoin; Dexamethasone and Panobinostat; Tretinoin and Panobinostat; Dexamethasone and Tretinoin and Calcitriol; and Dexamethasone and Tretinoin and Panobinostat.

34. The method of claim 33, wherein the priming agent is a combination of Dexamethasone and Panobinostat; or a combination of Tretinoin and Panobinostat.

35. The method of claim 11, wherein the priming agent is a priming-tuning agent which, in addition to priming, functions to suppress expression of an undesired target surface protein.

36. The method of claim 35, wherein the priming-tuning agent functions to suppress expression of at least one surface protein antagonizing T-cells.

37. The method of claim 36 wherein the priming-tuning agent functions to suppress expression of PD-L1.

38. The method of claim 11, further comprising the step of administering to the subject a priming-tuning agent which, in addition to priming, functions to suppress expression of an undesired target surface protein.

39. The method of claim 38, wherein the priming-tuning agent functions to suppress expression of at least one surface marker antagonizing T-cells.

40. The method of claim 39, wherein the priming-tuning agent functions to suppress expression of PD-L1.

41. The method of claim 38, wherein the priming-tuning agent is Panobinostat.

42. The method of claim 41, wherein Panobinostat is administered in combination with Dexamethasone or Tretinoin.

43. A method for identifying druggable therapeutic targets, comprising:
a. contacting a test-cancerous-cell, and a non-cancerous-control-cell, with one or more priming agents;
b. assaying marker expression on both the test-cancerous-cell and the non-cancerous-control-cell in response to the priming agent, and further assaying surface marker expression on a control-cancerous-cell that was not contacted with priming agents; and
c. identifying as a candidate therapeutic target any markers that have increased expression in the test-cancer-cell compared to both the control-cancerous-cell and the non-cancerous-control-cell.

44. The method of claim 43, wherein the marker expression is selected from extracellular or intracellular expression.

45. A method for identifying druggable targets, comprising:
a. contacting a test-cancerous-cell, and a non-cancerous-control-cell, with one or more priming agents; and
b. assaying surface marker expression on both the test-cancerous-cell and the non-cancerous-control-cell in response to the priming agent, and further assaying surface marker expression on a control-cancerous-cell that was not contacted with priming agents;
wherein increased surface marker expression of a particular target on the test-cancerous-cell compared to both the control-cancerous-cell and the non-cancerous-control-cell, identifies a druggable target.

46. A method for identifying priming agents comprising:
a. contacting a test-cancerous cell and a non-cancerous cell with a plurality of priming agent candidates;
b. assaying surface marker expression on both the test-cancerous cell and the non-cancerous cell in response to the priming agent candidates, and further assaying surface marker expression on a control-cancerous cell that was not contacted with priming agent candidates; and
c. identifying priming agent candidates that increase surface marker expression on the test-cancerous cell compared to both the control-cancerous cell and the non-cancerous-control cell as priming agents.

47. The method of claim 46, further comprising administering one or more priming agents identified to a subject in need.

48. The method of claim 47, wherein the subject in need has a proliferative disease.

49. The method of claim 48, wherein the proliferative disease is cancer.
